(19) United States
(12) Patent Application Publication
(10) Pub. No.: US 2012/0016230 A1
(43) Pub. Date: Jan. 19, 2012

(54) IMAGING APPARATUS, IMAGING SYSTEM, SURGICAL NAVIGATION SYSTEM, AND IMAGING METHOD

(75) Inventors: Koichiro Kishima, Kanagawa (JP); Tetsumasa Yonemitsu, Kanagawa (JP); Gakuho Fukushi, Tokyo (JP)

(73) Assignee: SONY CORPORATION, Tokyo (JP)

(21) Appl. No.: 13/178,155

(22) Filed: Jul. 7, 2011

(30) Foreign Application Priority Data
Jul. 13, 2010 (JP) 2010-158848

(51) Int. Cl. A61B 6/00

(52) U.S. Cl. 600/425

(57) ABSTRACT

An imaging apparatus includes: a first illumination unit to apply visible light to a subject having a fluorescent substance; a second illumination unit to apply excitation light to the subject so that fluorescence is generated from the fluorescent substance; an optical filter unit to cause the visible light and the fluorescence to pass therethrough, and shield the excitation light; an imaging unit including imaging elements to generate image signals, and an output unit to read the image signals from the imaging elements and output image information; an optical element to divide the visible light into component light beams, cause the divided light beams to be incident on the imaging elements, and cause the fluorescence to be incident on at least one imaging element; and a control means for alternately applying the visible light and the excitation light and alternately outputting image information of the visible light and the fluorescence.

### CROSS REFERENCES TO RELATED APPLICATIONS

The present application claims priority to Japanese Priority Patent Application JP 2010-158848 filed in the Japan Patent Office on Jul. 13, 2010, the entire content of which is hereby incorporated by reference.

### BACKGROUND

The present application relates to an imaging apparatus, an imaging system, a surgical navigation system, and an imaging method that are capable of capturing an image of a subject having a fluorescent pigment.

There has been known a technique of applying excitation light such as infrared light to an observation target to which a fluorescent pigment is given, and observing the observation target based on distribution or the like of the fluorescent pigment that emits light. As the fluorescent pigment used in this technique, for example, indocyanine green (ICG) is used. The ICG emits near infrared light of about 850 nm with respect to excitation light of about 800 nm. For , in the surgery of breast cancer, an image of a fluorescence phenomenon of the ICG given into a human body is captured and an infrared light image thereof is observed, and accordingly a position of a sentinel lymph node to be removed can be identified.

In an imaging apparatus disclosed in Japanese Patent Application Laid-open No. 2009-66121 (hereinafter, referred to as Patent Document 1), by light-receiving pixels to which a color filter is provided, a visible light image and an infrared light image of an observation target to which a fluorescent pigment is given are captured. Then, a combined image of the visible light image and the infrared light image is generated (paragraphs [0015] and [0017] and FIG. 3 of Patent Document 1).

### SUMMARY

In the case where the combined image of the visible light image and the infrared light image disclosed in Patent Document 1 is used in the surgery described above or the like, it is necessary to capture an infrared light image with high accuracy so as to precisely grasp the distribution or the like of the fluorescent pigment. Further, when an exposure time for capturing an infrared light image is long, an image of the situation of a tissue or the like during surgery is not captured in real-time, which makes it difficult to perform precise surgery.

In view of the circumstances as described above, it is desirable to provide an imaging apparatus, an imaging system, a surgical navigation system, and an imaging method that are capable of capturing an image of a subject having a fluorescent substance with high accuracy in a short exposure time.

According to an embodiment, there is provided an imaging apparatus including a first illumination unit, a second illumination unit, an optical filter unit, an imaging unit, an optical element, and a control means.

The first illumination unit is configured to apply visible light to a subject having a fluorescent substance.

The second illumination unit is configured to apply excitation light to the subject so that fluorescence is generated from the fluorescent substance.

The optical filter unit is configured to cause the visible light applied by the first illumination unit and the fluorescence generated from the fluorescent substance to pass therethrough, and shield the excitation light applied by the second illumination unit.

The imaging unit includes a plurality of imaging elements and an output unit. The plurality of imaging elements are capable of generating respective image signals based on incident light. The output unit reads the image signals from the plurality of imaging elements and outputs image information based on the read image signals.

The optical element is configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements.

The control means alternately applies the visible light and the excitation light and alternately outputs image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.

In the imaging apparatus, the imaging unit includes the plurality of imaging elements, and the image information of the visible light and the image information of the fluorescence are alternately output based on the image signals generated by the plurality of imaging elements. Therefore, the imaging unit with excellent image-capturing sensitivity allows the image information of the visible light and the image information of the fluorescence to be output in a short exposure time. Further, a highly accurate visible light image and fluorescence image can be obtained. As a result, an image of a subject having a fluorescent substance can be captured with high accuracy in a short exposure time.

The output unit may be capable of reading the image signals from the respective imaging elements in an interlace system and outputting first field image information and second field image information that constitute frame image information based on the read image signals. In this case, the control means may output the image information of the visible light as the first field image information and output the image information of the fluorescence as the second field image information.

In the imaging apparatus, the image information of the visible light is output as the first field image information, and the image information of the fluorescence is output as the second field image information. Accordingly, a combined image of the visible light image and the fluorescence image can be obtained with ease.

The plurality of imaging elements may be complementary metal oxide semiconductor (CMOS) image sensors that generate the image signals in a rolling shutter system. In this case, the control means may stop applying the visible light and the excitation light during a time in which the image signals are being read from the CMOS image sensors by the output unit.

In the imaging apparatus, during a time in which the image signals are being read from the CMOS sensors, the application of the visible light and the excitation light is stopped. Accordingly, the imaging unit having the CMOS sensors driven in the rolling shutter system can appropriately output the image information of the visible light and the image information of the fluorescence.

The subject may have a first fluorescent substance to generate first fluorescence by first excitation light being applied, and a second fluorescent substance to generate second fluorescence by second excitation light being applied, the second excitation light being different from the first excitation light.

In this case, the second illumination unit may include a first excitation-light illumination unit to apply the first excitation light, and a second excitation-light illumination unit to apply the second excitation light.

Further, the optical filter unit may include a first filter to cause the visible light and the first fluorescence to pass therethrough and shield the first excitation light, and a second filter to cause the visible light and the second fluorescence to pass therethrough and shield the second excitation light.

Further, the optical element may divide the visible light having passed through one of the first optical filter and the second optical filter into a plurality of component light beams, cause the divided component light beams to be incident on the respective imaging elements, and cause the first fluorescence and the second fluorescence to be incident on at least one of the plurality of imaging elements.

Further, the control means may switch between the visible light, the first excitation light, and the second excitation light to be applied, alternately switch, based on a timing of the switching, between the first optical filter and the second optical filter, and alternately output the image information of the visible light, image information of the first fluorescence, and image information of the second fluorescence, the image information of the first fluorescence being based on at least one of the image signals read from the at least one of the imaging elements on which the first fluorescence is incident, the image information of the second fluorescence being based on at least one of the image signals read from the at least one of the imaging elements on which the second fluorescence is incident.

By the imaging apparatus, for example, an image of a subject having different types of fluorescent substances can be captured with high accuracy in a short exposure time.

According to an embodiment, there is provided an imaging system including an illumination apparatus, an imaging apparatus, and a control means.

The illumination apparatus includes a first illumination unit configured to apply visible light to a subject having a fluorescent substance, and a second illumination unit configured to apply excitation light to the subject so that fluorescence is generated from the fluorescent substance.

The imaging apparatus includes an optical filter unit, an imaging unit, and an optical element.

The optical filter unit is configured to cause the visible light applied by the first illumination unit and the fluorescence generated from the fluorescent substance to pass therethrough, and shield the excitation light applied by the second illumination unit.

The imaging unit includes a plurality of imaging elements and an output unit. The plurality of imaging elements are capable of generating respective image signals based on incident light. The output unit reads the image signals from the plurality of imaging elements and outputs image information based on the read image signals.

The optical element is configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements.

The control means alternately applies the visible light and the excitation light and alternately outputs image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.

According to an embodiment, there is provided a surgical navigation system including a display, an illumination apparatus, an imaging apparatus, and a control means.

The illumination apparatus includes a first illumination unit configured to apply visible light to an operated portion to which a fluorescent substance is given, and a second illumination unit configured to apply excitation light to the operated portion so that fluorescence is generated from the fluorescent substance.

The imaging apparatus includes an optical filter unit, an imaging unit, and an optical element.

The optical filter unit is configured to cause the visible light applied by the first illumination unit and the fluorescence generated from the fluorescent substance to pass therethrough, and shield the excitation light applied by the second illumination unit.

The imaging unit includes a plurality of imaging elements and an output unit. The plurality of imaging elements are capable of generating respective image signals based on incident light. The output unit reads the image signals from the plurality of imaging elements and outputs image information based on the read image signals.

The optical element is configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements.

The control means alternately applies the visible light and the excitation light, alternately outputs image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident, and displays a visible light image and a fluorescence image on the display based on the output image information of the visible light and the output image information of the fluorescence.

According to an embodiment, there is provided an imaging method including alternately applying, to a subject having a fluorescent substance, visible light and excitation light for generating fluorescence from the fluorescent substance.

By an optical filter unit, the visible light applied to the subject and the fluorescence generated from the fluorescent substance are caused to pass therethrough, and the excitation light applied to the subject is shielded.

The visible light having passed through the optical filter unit is divided into a plurality of component light beams, the divided component light beams are caused to be incident on a plurality of imaging elements capable of generating respective image signals based on incident light, and the fluorescence is caused to be incident on at least one of the plurality of imaging elements, to thereby alternately output image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.

As described above, according to the embodiments, an image of a subject having a fluorescent substance can be captured with high accuracy in a short exposure time.

Additional features and advantages are described herein, and will be apparent from the following Detailed Description and the figures.

### BRIEF DESCRIPTION OF THE FIGURES

FIG. 1 is a schematic diagram showing an of a structure of an imaging system according to a first embodiment;

FIG. 2 is a block diagram showing a functional structure of the imaging system shown in FIG. 1;

FIG. 3 is a schematic diagram showing an of a structure of a camera shown in FIG. 1;

FIG. 4 is a graph showing optical characteristics of an optical filter unit included in the camera shown in FIG. 3;

FIG. 5 is a diagram schematically showing a structure of a sensor unit shown in FIG. 3;

FIG. 6 is a diagram schematically showing an imaging element included in the sensor unit shown in FIG. 5;

FIG. 7 is a schematic graph showing optical characteristics of a visible-light filter shown in FIG. 1;

FIG. 8 is a schematic graph showing optical characteristics of an excitation-light filter shown in FIG. 1;

FIG. 9 is a schematic diagram showing a structure of a PC (Personal Computer) as an of a device functioning as a magnification controller unit of the first embodiment;

FIG. 10 is a schematic diagram showing a visible light image captured by the imaging system according to the first embodiment;

FIG. 11 is a schematic diagram showing a fluorescence image captured by the imaging system according to the first embodiment;

FIG. 12 is a schematic diagram showing an imaging element in the case where one imaging element is used to capture a color image;

FIG. 13 is a schematic diagram showing a combined image obtained by combining the visible light image and the fluorescence image captured by the imaging system according to the first embodiment;

FIG. 14 is a schematic diagram showing another of the fluorescence image captured by the imaging system according to the first embodiment;

FIG. 15 is a schematic diagram showing a combined image obtained by combining the visible light image and the fluorescence image shown in FIG. 14 that are captured by the imaging system according to the first embodiment;

FIG. 16 is a diagram for describing an operation in which image information is output by a circuit unit in a camera according to a second embodiment;

FIG. 17 is a schematic diagram showing a combined image generated by an imaging system according to the second embodiment;

FIG. 18 is a schematic diagram showing a combined image generated by the imaging system according to the second embodiment;

FIG. 19 is a timing chart showing an of an application timing of the visible light and the excitation light and a read timing of an image signal from a CMOS sensor in a third embodiment;

FIG. 20 is a schematic diagram showing an of a structure of an imaging system according to a fourth embodiment;

FIG. 21 is a schematic diagram showing an of a structure of a surgical navigation system according to a fifth embodiment;

FIG. 22 is a schematic diagram showing an of a structure of the surgical navigation system according to the fifth embodiment;

FIG. 23 is a schematic diagram showing an of a structure of an endoscope as an imaging apparatus according to a sixth embodiment;

FIG. 24 is a schematic diagram showing an of a structure of the endoscope as the imaging apparatus according to the sixth embodiment; and

FIG. 25 is a schematic diagram showing another of the structure of the camera shown in FIG. 3.

### DETAILED DESCRIPTION

Embodiments of the present application will be described below in detail with reference to the drawings.

Hereinafter, embodiments will be described with reference to the drawings.

### First Embodiment

Structure of Imaging System

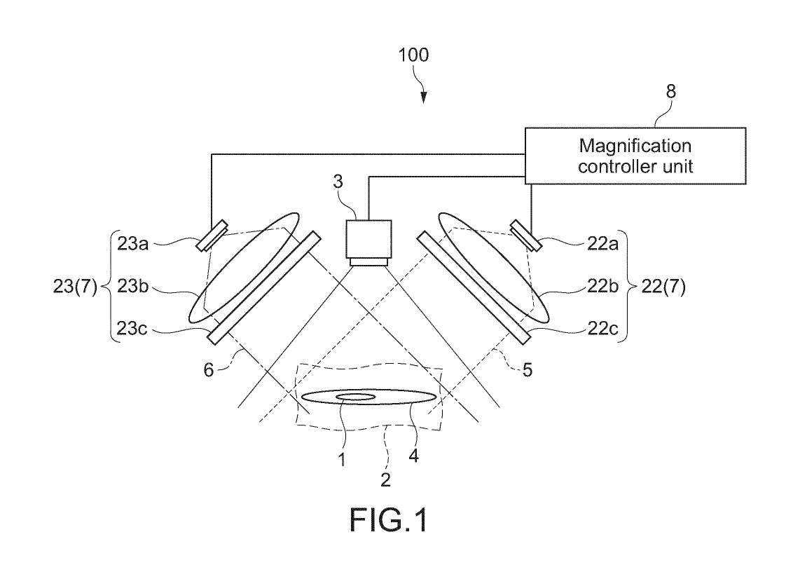

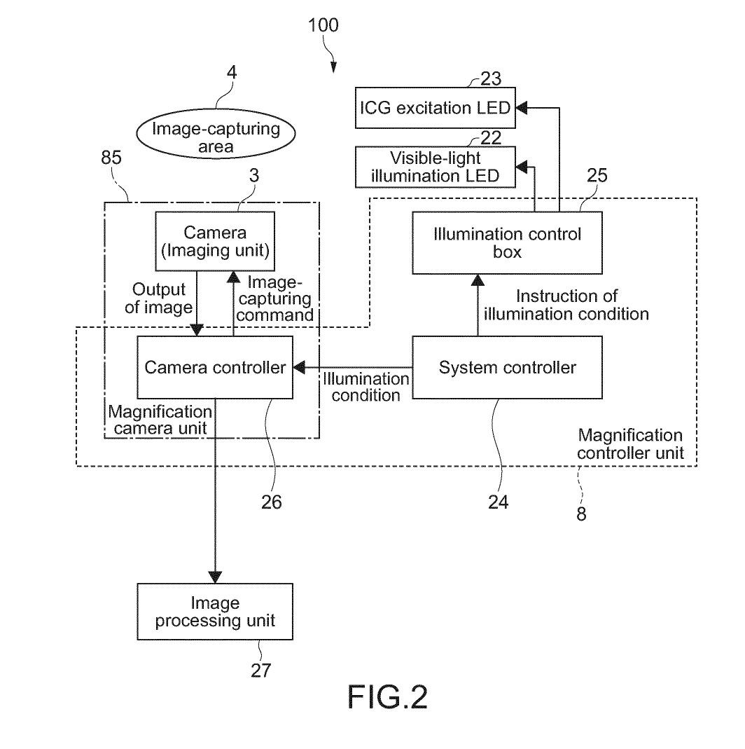

FIG. 1 is a schematic diagram showing an of a structure of an imaging system according to a first embodiment. FIG. 2 is a block diagram showing a functional structure of the imaging system shown in FIG. 1.

An imaging system 100 of this embodiment is a system for capturing an image of a subject 2 having a fluorescent substance 1, and includes a camera 3 serving as an imaging apparatus, an illumination apparatus 7 that applies visible light 5 and excitation light 6 to an image-capturing area 4 whose image is captured by the camera 3, and a magnification controller unit 8 serving as a control means for controlling the camera 3 and the illumination apparatus 7. In this embodiment, used as the fluorescent substance 1 contained in the subject 2 is ICG (Indocyanine green) in which the center value of a fluorescence excitation wavelength is about 786 nm and a fluorescence emission wavelength is about 845 nm.

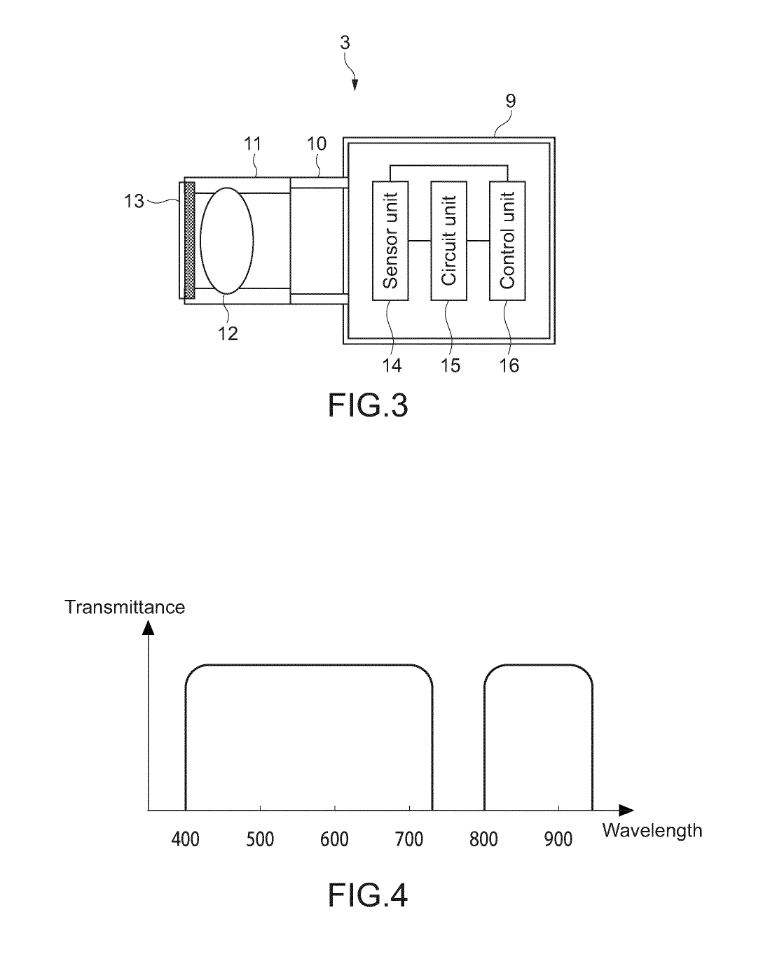

FIG. 3 is a schematic diagram showing an of a structure of the camera 3. FIG. 4 is a graph showing optical characteristics of an optical filter unit included in the camera 3.

The camera 3 includes a main body 9, a camera mount 10, and a lens unit 11 detachably mounted to the camera mount 10. The lens unit 11 includes a lens 12 for image capturing and an optical filter 13 serving as an optical filter unit arranged in front of the lens 12.

The optical filter 13 causes visible light containing component light beams of red (R), green (G), and blue (B) and fluorescence emitted from the exited ICG to pass therethrough. Further, the optical filter 13 shields excitation light applied to the subject 2. Specifically, as shown in the graph of FIG. 4, the optical filter 13 causes visible light in a visible light band having the wavelength range of about 400 nm to about 740 nm and fluorescence in the wavelength range of about 800 nm or more to pass therethrough. A transmittance is 90% or more, for . Excitation light in a fluorescence excitation light band having the wavelength range of about 750 nm to about 790 nm is shielded at an optical density (OD) of 2 or more, that is, at a transmittance of 1% or less. Since the optical filter 13 is arranged in front of the lens 12, for , when an image of a fluorescent substance different from ICG is captured, an optical filter that is based on wavelengths of fluorescence and excitation light of that fluorescent substance can be attached to the lens unit 11 easily.

The main body 9 includes a sensor unit 14 that can generate an analog image signal based on an intensity of incident light, and a circuit unit 15 serving as an output means for reading the image signal from the sensor unit 14 and outputting digital image information based on the image signal. Further, the main body 9 includes a control unit 16 that has a main memory or the like constituted of, for example, a CPU (Central Processing Unit), a ROM (Read Only Memory), or a RAM (Random Access Memory), and controls the whole of the camera 3 including the sensor unit 14 and the circuit unit 15. The sensor unit 14 and the circuit unit 15, or the sensor unit 14, the circuit unit 15, and the control unit 16 constitute an imaging unit according to this embodiment.

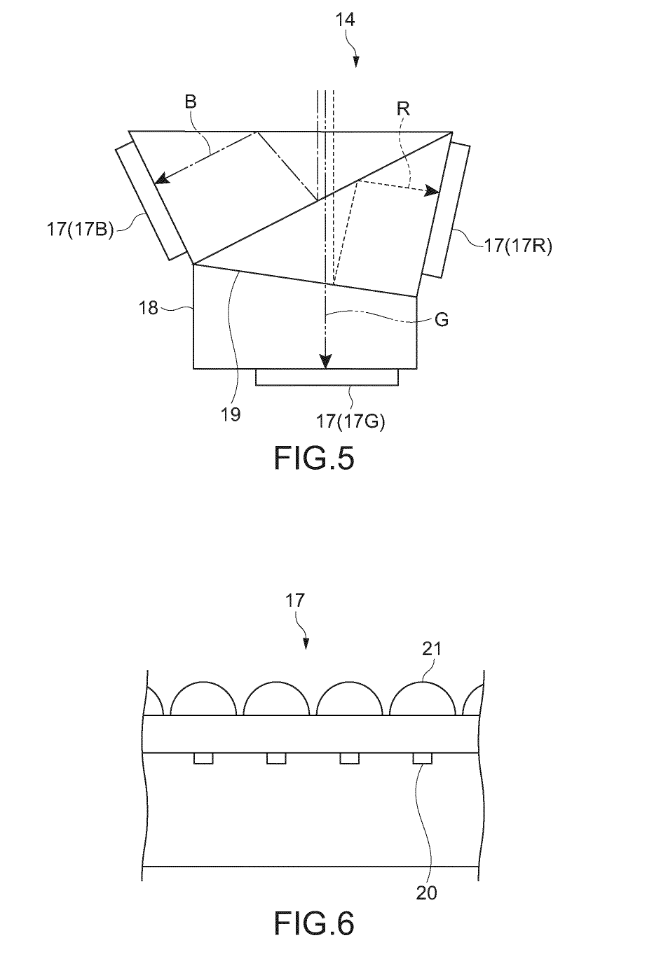

FIG. 5 is a diagram schematically showing an of a structure of the sensor unit 14. FIG. 6 is a diagram schematically showing an imaging element included in the sensor unit 14 shown in FIG. 5.

The sensor unit 14 includes a plurality of imaging elements 17 and in this embodiment, three imaging elements 17 (17R, 17G, 17B) are arranged in the sensor unit 14 as shown in FIG. 5. As the imaging element 17, for example, a CCD (Charge Coupled Device) sensor or a CMOS (Complementary Metal Oxide Semiconductor) sensor is used.

Further, the sensor unit 14 includes a spectral prism 18 serving as an optical element that divides the visible light, which has passed through the optical filter 13, into a plurality of component light beams. In the spectral prism 18, by coating surfaces 19 at prism boundaries, light reflected on the boundary and a light that passes therethrough are defined. In this embodiment, visible light is divided into three component light beams of R, G, and B by the spectral prism 18, and the component light beams R, G, and B are incident on the three imaging elements 17R, 17G, and 17B, respectively. Further, fluorescence that has passed through the optical filter 13 (not shown) is incident on at least one of the three imaging elements 17 by the spectral prism 18. As the spectral prism 18, for , a dichroic prism is used.

As shown in FIG. 6, each of the imaging elements 17 includes a plurality of light-receiving elements 20 corresponding to pixels of an image to be generated, and the component light beams R, G, and B are incident on the light-receiving elements 20 via on-chip lenses 21. Then, the component light beams R, G, and B are subjected to photoelectric conversion by the light-receiving elements 20 so that image signals of the respective component light beams R, G, and B are output.

The circuit unit 15 includes an analog signal processing circuit, an A/D converter circuit, a digital signal processing circuit, and various processing circuits such as a sensor unit driver circuit (not shown). The circuit unit 15 reads the image signals from the plurality of imaging elements 17 and outputs image information as frame image information based on the read image signals. A frame rate of the output frame image information is 30 fps (frame per second), for example.

As shown in FIG. 1, the illumination apparatus 7 includes a visible-light illumination unit 22 serving as a first illumination unit, and an excitation-light illumination unit 23 serving as a second illumination unit.

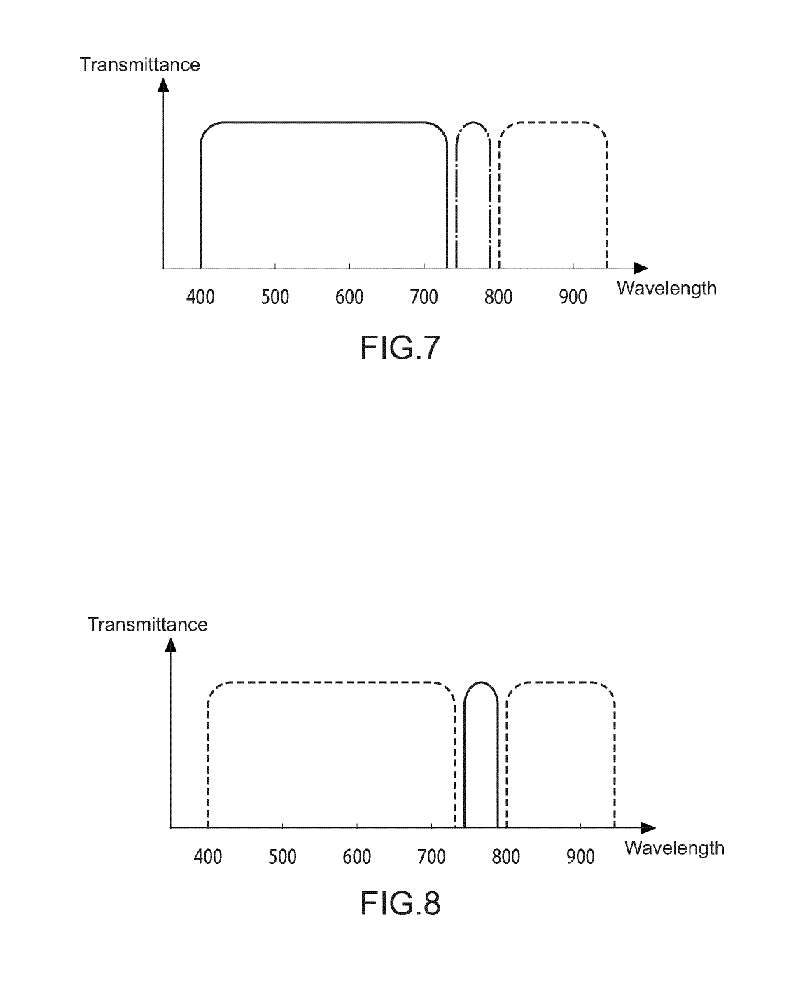

The visible-light illumination unit 22 includes a visible-light illumination 22a, a lens 22b, and a visible-light filter 22c. The visible-light illumination 22a has, for example, a white LED (Light Emitting Diode) or a visible-light illumination LED constituted of LEDs of the respective colors. The lens 22b effectively guides the visible light 5 emitted from the visible-light illumination 22a to the image-capturing area 4. FIG. 7 is a schematic graph showing optical characteristics of the visible-light filter 22c. As shown in the graph, the visible-light filter 22c causes the visible light 5 in the wavelength range of about 400 nm to about 740 nm to pass therethrough.

The excitation-light illumination unit 23 includes an excitation-light illumination 23a, a lens 23b, and an excitation-light filter 23c. The excitation-light illumination 23a includes, for example, an ICG excitation LED. The lens 23b effectively guides the excitation light emitted from the excitation-light illumination 23a to the image-capturing area 4. The excitation-light filter 23c is used in order that the excitation light 6 applied from the excitation-light illumination 23a does not contain a component of a transmission wavelength band of the optical filter 13 provided to the camera 3.

FIG. 8 is a schematic graph showing optical characteristics of the excitation-light filter 23c. The excitation-light filter 23c of this embodiment causes the excitation light 6 in the wavelength range of about 755 nm to about 785 nm to pass therethrough. In other words, a transmission wavelength band of the excitation-light filter 23c is narrowed by 5 nm from each of a lower limit value and an upper limit value of the shield wavelength band of the optical filter 13. Accordingly, the excitation light 6 applied from the excitation-light illumination unit 23 can be satisfactorily prevented from being incident on the sensor unit 14 inside the camera 3. As a result, a visible light image and a fluorescence image can be captured with high accuracy. It should be noted that the transmission wavelength band of the excitation-light filter 23c may be made smaller. Alternatively, the transmission wavelength band of the excitation-light filter 23c and the shield wavelength band of the optical filter 13 may be substantially the same.

The structure of the visible-light illumination unit 22 that applies the visible light 5 to the subject 2, and the structure of the excitation-light illumination unit 23 that applies the excitation light 6 thereto are not limited to those described in this embodiment. For example, a light source other than the LED may be used. Further, a structure in which the lens 22b or 23b, the visible-light filter 22c, or the like is not used may be adopted.

As shown in FIG. 2, the magnification controller unit 8 includes a system controller 24, an illumination control box 25, and a camera controller 26.

The system controller 24 outputs instruction information on an illumination condition to the illumination control box 25. As the illumination condition, an application timing of the visible light and the excitation light, an application time, an intensity of the visible light and the excitation light, or the like is included.

The illumination control box 25 drives the visible-light illumination unit 22 including the visible-light illumination LED, and the excitation-light illumination unit 23 including the ICG excitation LED based on the instruction information output from the system controller 24.

Further, the system controller 24 outputs the above-mentioned illumination condition as information to the camera controller 26. The camera controller 26 outputs image-capturing command information to the control unit 16 in the camera 3 shown in FIG. 3 based on the illumination condition information output from the system controller 24. Furthermore, the camera controller 26 outputs the image information output from the camera 3 to an image processing unit 27 shown in FIG. 2.

The image processing unit 27 performs various types of image processing such as combining processing on the image information output from the camera controller 26.

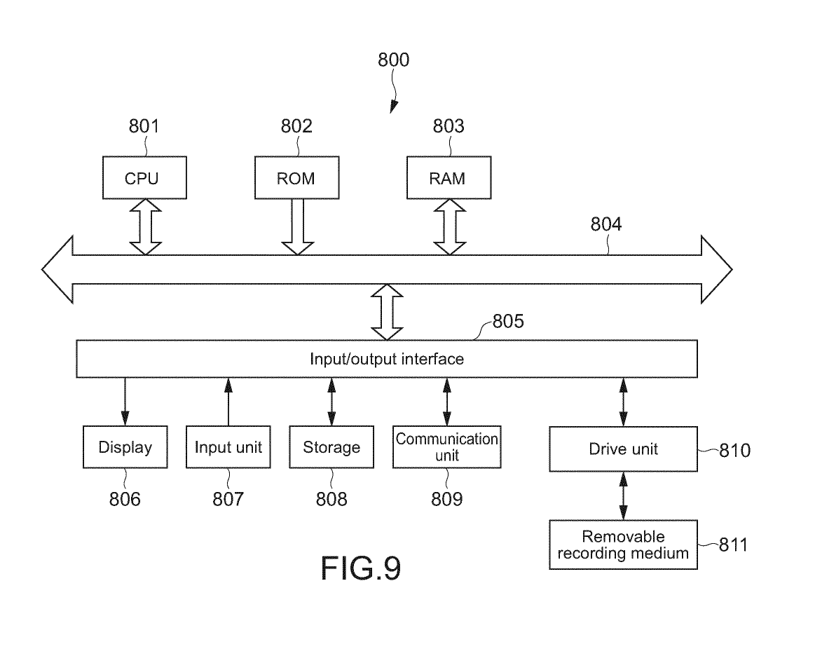

FIG. 9 is a schematic diagram showing a structure of a PC (Personal Computer) as an of a device functioning as the magnification controller unit 8 of this embodiment. It should be noted that in this embodiment, the PC also functions as the image processing unit described above.

A PC 800 includes a CPU 801, a ROM 802, a RAM 803, an input/output interface 805, and a bus 804 that connects those components.

To the input/output interface 805, a display 806, an input unit 807, a storage 808, a communication unit 809, a drive unit 810, and the like are connected.

The display 806 is a display device in which liquid crystal, EL (Electro-Luminescence), or CRT (Cathode Ray Tube) is used, for example.

The input unit 807 is, for , a pointing device, a keyboard, a touch panel, or other operation apparatus. In the case where the input unit 807 includes a touch panel, the touch panel may be integrated with the display 806.

The storage 808 is a nonvolatile storage device such as an HDD (Hard Disk Drive), a flash memory, or other solid-state memory.

The drive unit 810 is a device capable of driving a removable recording medium 811 such as an optical recording medium, a floppy (registered trademark) disk, a magnetic recording tape, or a flash memory. In contrast to this, the storage 808 is used as a built-in device of the PC 800 that mainly drives non-removable recording media in many cases.

The communication unit 809 is a modem, a router, or other communication equipment that is capable of connecting to a LAN (Local Area Network), a WAN (Wide Area Network), or the like and used for communicating with other devices. The communication unit 809 may perform wired or wireless communication. The communication unit 809 may be used separately from the PC 800 in many cases.

The PC 800 described above operates as the magnification controller unit 8 so that various types of data processing are performed. The data processing performed by the PC 800 is realized in cooperation with software stored in the storage 808, the ROM 802, or the like and hardware resources of the PC 800. Specifically, the CPU 801 loads a program constituting the software, which is stored in the storage 808, the ROM 802, or the like, to the RAM 803 and executes the program, and accordingly various types of data processing are realized.

It should be noted that as the magnification controller unit 8 and the image processing unit 27, instead of the PC 800, a dedicated control device may be used. Further, different devices may be used as the magnification controller unit 8 and the image processing unit 27.

Operation of Imaging System

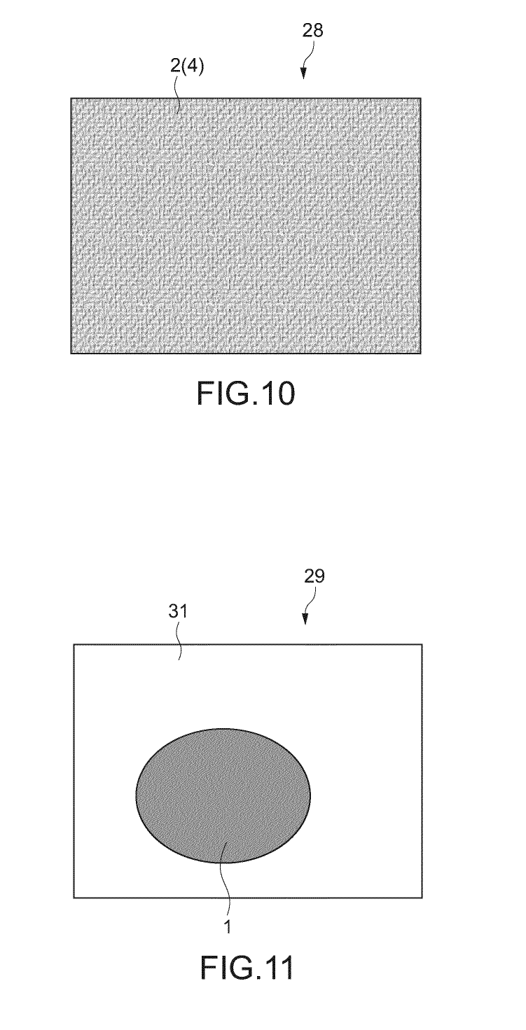

Description will be given on the operation of the imaging system 100 of this embodiment. FIG. 10 is a schematic diagram showing a visible light image captured by the imaging system of this embodiment. FIG. 11 is a schematic diagram showing a fluorescence image captured by the imaging system of this embodiment.

The drive of the visible-light illumination unit 22 and excitation-light illumination unit 23 is controlled by the magnification controller unit 8 so that the visible light 5 and the excitation light 6 are alternately applied to the subject 2 arranged in the image-capturing area 4. In this embodiment, the application of the visible light 5 and that of the excitation light 6 are switched per 1/30 seconds in accordance with the frame rate described above. Further, the drive of the camera 3 is controlled by the magnification controller unit 8 so that visible-light image information and fluorescence image information are each output alternately as frame image information, based on an application timing of the visible light 5 and the excitation light 6 as follows.

When the visible light 5 is applied to the subject 2 by the visible-light illumination unit 22, visible light reflected on the subject 2 passes through the optical filter 13 having the optical characteristics shown in FIG. 4 to be incident on the sensor unit 14. By the spectral prism 18 of the sensor unit 14, the visible light is divided into three component light beams of R, G, and B and the component light beams R, G, and B are incident on the three imaging elements 17R, 17G, and 17B, respectively. Then, image signals of the component light beams R, G, and B are generated by the imaging elements 17R, 17G, and 17B, respectively. The generated image signals of the component light beams R, G, and B are each read by the circuit unit 15 and visible-light image information is output based on each of the image signals. Based on the visible-light image information, as shown in FIG. 10, a visible light image 28 that is a color image of the subject 2 (image-capturing area 4) is captured.

When the excitation light 6 is applied to the subject 2 by the excitation-light illumination unit 23, fluorescence from the fluorescent substance 1 included in the subject 2 passes through the optical filter 13 to be incident on the sensor unit 14. In this embodiment, the fluorescence having a wavelength of about 850 nm is incident on the imaging element 17G on which the component light beam G is mainly incident, and an image signal is thus generated. The image signal is read from the imaging element 17G by the circuit unit 15, and fluorescence image information is output based on the image signal. Based on the fluorescence image information, as shown in FIG. 11, a fluorescence image 29 that is a color image of the fluorescent substance 1 is captured. It should be noted that a color of the fluorescent substance 1 within the fluorescence image 29 is not limited to green. The color of the fluorescent substance 1 may be set as appropriate by the circuit unit 15 and fluorescence image information thereof may be output. Alternatively, the color of the fluorescent substance 1 may be set as appropriate by the image processing unit 27.

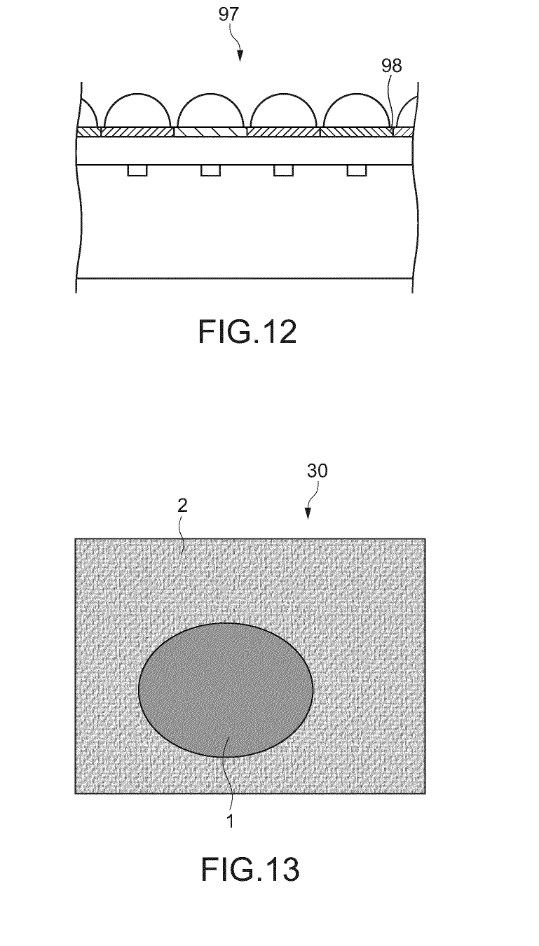

For example, in the case where one imaging element 97 is used solely to capture a color image, as shown in FIG. 12, a color filter 98 has to be provided to the imaging element 97. In this case, since light that is incident on the imaging element 97 is absorbed by the color filter 98, the efficiency of light absorption is low.

In contrast to this, a color filter is not used in this embodiment, and the on-chip lens 21 shown in FIG. 6 is not provided with a material to absorb light. In addition, the spectral prism 18 is used to divide visible light by means of characteristics of reflection and transmission on the coating surface 19, and a material to absorb light is not also used for the spectral prism 18. Therefore, the visible light and the fluorescence that are incident on the sensor unit 14 according to this embodiment are imaged with high efficiency, that is, with high sensitivity, with the result that the visible-light image information and the fluorescence image information can be output in a short exposure time. Further, a highly accurate visible light image 28 and fluorescence image 29 can be obtained. As a result, an image of the subject 2 including the fluorescent substance 1 can be captured with high accuracy in a short exposure time.

Since the application of the visible light and that of the excitation light are switched per 1/30 seconds, a period of time in which illumination light is dark (excitation light application time) is very short. Therefore, for example, in the case where surgery or the like is performed using this imaging system, the field of view of the surgeon does not substantially become dark. Further, since the visible-light image information and the fluorescence image information are alternately output at a frame rate of 30 fps, an image of the subject 2 including the fluorescent substance 1 can be captured in real-time. By those pieces of information, the surgeon can perform precise surgery. Furthermore, since an image of the subject 2 can be captured in a short exposure time, the influence of heat generated by the application of the visible light and excitation light on the subject 2 can be suppressed.

The visible-light image information and fluorescence image information output by the camera 3 are output to the image processing unit 27. At this time, information for identifying the visible-light image information and the fluorescence image information is given to each piece of the image information. Further, information on the above-mentioned image-capturing condition used when the pieces of image information are generated may be given.

The visible light image 28 and the fluorescence image 29 are combined by the image processing unit 27. FIG. 13 is a schematic diagram showing a combined image 30 obtained by combining the visible light image 28 and the fluorescence image 29.

The combining processing of the visible light image 28 and the fluorescence image 29 may adopt various types of combining processing as appropriate, such as adding data of pixels of both the images. An example of the combining processing will be described below.

For example, assuming that an area 31 of the fluorescence image 29 shown in FIG. 11 is transparent, in which the fluorescent substance 1 is not located, such a fluorescence image 29 may be combined with the visible light image 28 shown in FIG. 10. Accordingly, a combined image 30 shown in FIG. 13 can be generated through simple processing.

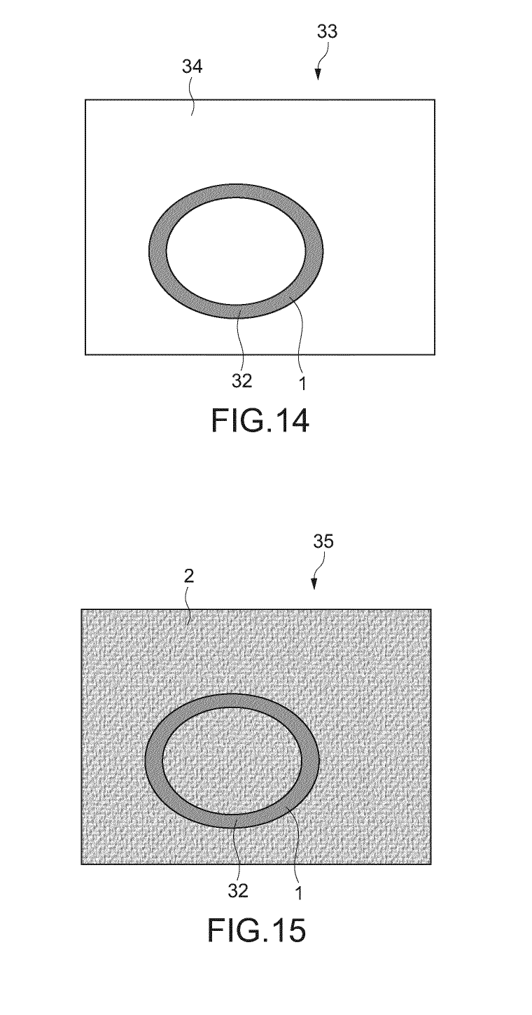

Further, an outline portion 32 of the fluorescent substance 1 may be detected from the fluorescence image 29 shown in FIG. 11 by image processing such as differential processing, and that image may be generated as an outline image 33 as shown in FIG. 14. Assuming that an area 34 of the outline image 33 is transparent, in which the outline portion 32 of the fluorescent substance 1 is not located, the visible light image 28 and the outline image 33 may be combined. Accordingly, a combined image 35 as shown in FIG. 15 can be generated. For example, in the case where a portion in which the fluorescent substance 1 is located is intended to be removed in surgery of a human body, the use of the combined image 35 shown in FIG. 15 makes the removal operation easy.

As described above, in this embodiment, the PC provided separately from the camera 3 functions as the image processing unit 27. However, it may be possible to provide a block to process an image in the camera 3, perform the image processing described above in the camera 3, and output visible-light image information and fluorescence image information that have been subjected to the image processing.

Further, only the visible-light illumination unit 22 is driven and a visible light image of the subject 2 is captured, with the result that the imaging system 100 of this embodiment can be used as a recording system to record a status of the subject 2.

### Second Embodiment

Description will be given on an imaging system according to a second embodiment. In the following description, the same structure and action as those of the imaging system 100 described in the first embodiment will not be described or simply described.

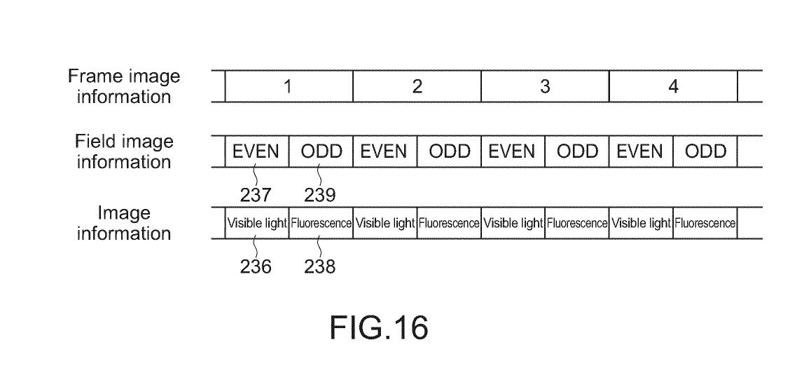

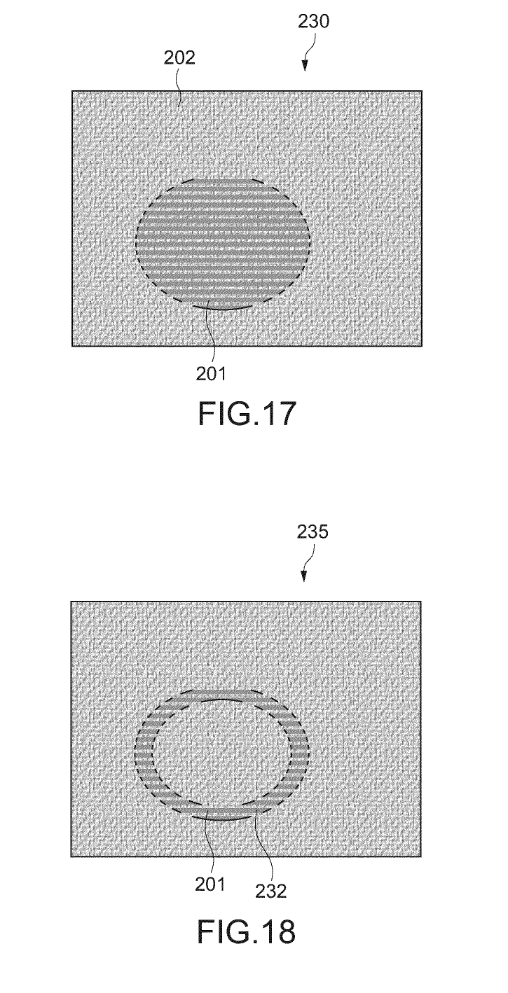

FIG. 16 is a diagram for describing an operation in which image information is output by a circuit unit in a camera according to this embodiment. FIGS. 17 and 18 are schematic diagrams each showing a combined image generated by an imaging system according to this embodiment.

In this embodiment, image signals can be read from respective imaging elements by the circuit unit in the camera in an interlace system. Then, even field image information as first field image information and odd field image information as second field image information that constitute a frame image are output. A field rate of each field image information is 60 fps, that is, a frame rate of the frame image information is 30 fps.

The application of the visible light and that of the excitation light to the subject 2 are switched per 1/60 seconds in accordance with the field rate described above. As shown in FIG. 16, by the circuit unit, visible-light image information 236 is output as even field image information 237, and fluorescence image information 238 is output as odd field image information 239. Accordingly, as shown in FIG. 17, a combined image 230 can be easily obtained in which a visible light image of a subject 202 and a fluorescence image of a fluorescent substance 201 are combined without being subjected to image combining processing performed by the image processing unit. Further, alignment of a visible light image and a fluorescence image when both the images are combined is unnecessary.

Furthermore, it may be possible to process a fluorescence image in the camera and generate an outline image that is an image of an outline portion 232 of the fluorescent substance 201, which has been described in the first embodiment with reference to FIG. 14. Then, information of the outline image may be output as the odd field image information 239 to generate a combined image 235 as shown in FIG. 18.

It should be noted that the field rate of the field image information 237 and 239 to be output can be set as appropriate by controlling an application timing of illumination light of each of the visible-light illumination unit and the excitation-light illumination unit, and a read timing of the image signal from the imaging element by the circuit unit.

As described in the first embodiment described above, the visible light image and the fluorescence image can be captured with high accuracy in a short exposure time, with the result that both the images can be captured at a field rate of 60 fps as in this embodiment.

### Third Embodiment

Description will be given on an imaging system according to a third embodiment. In this embodiment, three CMOS sensors that generate image signals in a rolling shutter system are each used as an imaging element included in an imaging unit of a camera.

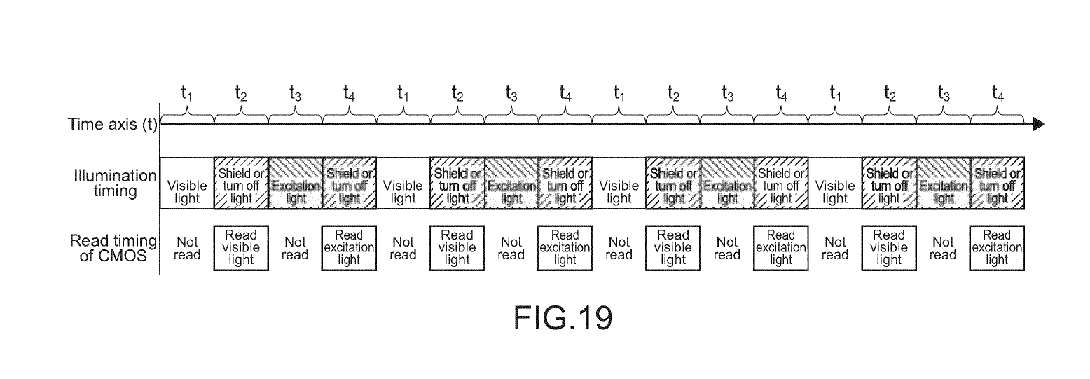

FIG. 19 is a timing chart showing an of an application timing of visible light and excitation light, and a read timing of the image signals from the CMOS sensors in this embodiment.

As shown in FIG. 19, visible light is applied to a subject by a visible-light illumination unit at a time t1. The visible light is incident on the CMOS sensors and charge is accumulated in light-receiving elements of the CMOS sensors. At a time t2, the accumulated charge is read for each line sequentially as an image signal corresponding to the visible light by an output unit in the camera. During the time t2 in which image signals are being read for each line sequentially, the application of the visible light and the excitation light is stopped.

When the read of image signals is ended at the time t2, excitation light is applied to the subject by the excitation-light illumination unit at a time t3. The fluorescence generated from the fluorescent substance is incident on the CMOS sensors, and charge is accumulated in the light-receiving elements of the CMOS sensors. At a time t4, the charge that is based on the fluorescence accumulated by the output unit in the camera is read for each line sequentially as an image signal. During the time t4 in which image signals are being read for each line sequentially, the application of the visible light and the excitation light is stopped.

The application time t1 of the visible light, the application time t3 of the excitation light, and the read times t2 and t4 of image signals are determined on one frame basis, for example. In other words, the visible light is applied in the first frame (time t1), and the application is stopped in the second frame (t2). The excitation light is applied in the third frame (time t3), and the application is stopped in the fourth frame (t4).

In this manner, an image signal corresponding to one frame may be read in a time corresponding to two frames. However, the application times t1 and t3 of the illumination light and the read times t2 and t4 of the image signals may be longer or shorter than a time for one frame. Further, the times t1 to t4 may be set to be different from one another.

As described above, in the imaging system according to this embodiment, during a time in which an image signal is being read from the CMOS sensors by the output unit in the camera (times t2 and t4), the application of the visible light and the excitation light is stopped. Therefore, no charge is accumulated in the CMOS sensors in the times t2 and t4. Accordingly, the influence of the rolling shutter system cab be prevented, such as a case where the upper half area of one frame image is an visible light image and the lower half area thereof is a fluorescence image due to a deviation between a read time and an exposure timing for each line, for example. In other words, visible-light image information and fluorescence image information can be output appropriately by a sensor unit and a circuit unit (imaging unit) that include the CMOS sensors driven by the rolling shutter system.

It should be noted that as shown in FIG. 19, instead of the stop of the application of the visible light and the fluorescence, the visible light and the fluorescence may be shielded by a light-shielding filter or the like during a time in which an image signal is being read from the CMOS sensor by the output unit. Further, the stop of the application of the visible light and the fluorescence and the shield of the visible light and the fluorescence by a light-shielding filter or the like may be used in combination.

### Fourth Embodiment

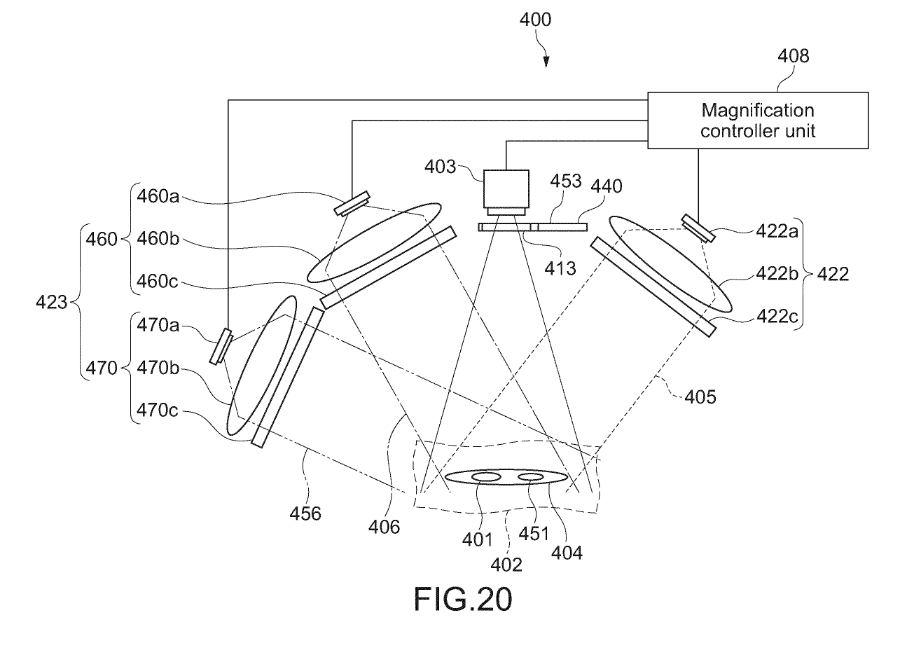

FIG. 20 is a schematic diagram showing an of a structure of an imaging system according to a fourth embodiment. In an imaging system 400 according to this embodiment, as shown in FIG. 20, an image of a subject 402 (image-capturing area 404) including two types of fluorescent substances of a first fluorescent substance 401 and a second fluorescent substance 451 can be captured. The first fluorescent substance 401 is excited when first excitation light 406 is applied thereto, and first fluorescence (not shown) is generated. The second fluorescent substance 451 is excited when second excitation light 456 is applied thereto, and second fluorescence (not shown) is generated. In this embodiment, the first and second excitation light 406 and 456 and the first and second fluorescence are near-infrared light similarly to the ICG described above.

As shown in FIG. 20, the imaging system 400 includes a visible-light illumination unit 422 and an excitation-light illumination unit 423. The visible-light illumination unit 422 includes a visible-light illumination 422a, a lens 422b, and a visible-light filter 422c.

The excitation-light illumination unit 423 includes a first excitation light source unit 460 as a first excitation-light illumination unit for generating the first excitation light 406, and a second excitation light source unit 470 as a second excitation-light illumination unit for generating the second excitation light 456. The first excitation light source unit 460 includes an excitation-light illumination 460a, a lens 460b, and an excitation-light filter 460c. The second excitation light source unit 470 includes an excitation-light illumination 470a, a lens 470b, and an excitation-light filter 470c.

Further, a camera 403 of the imaging system 400 according to this embodiment includes a filter changer 440 as an optical filter unit. The filter changer 440 includes a first optical filter 413 used for capturing an image of the first fluorescent substance 401, and a second optical filter 453 used for capturing an image of the second fluorescent substance 451. The first optical filter 413 causes the visible light and the first fluorescence generated from the excited first fluorescent substance 401 to pass therethrough and shields the first excitation light 406 applied to the subject 402. The second optical filter 453 causes the visible light and the second fluorescence generated from the excited second fluorescent substance 451 to pass therethrough and shields the second excitation light 456 applied to the subject 402.

The filter changer 440 may be formed integrally with the camera 403, or may be provided in front of the camera 403 separately from the camera 403. The drive of the filter changer 440 is controlled by a magnification controller unit 408.

The visible light 405, the first excitation light 406, and the second excitation light 456 are switched for application by the magnification controller unit 408. Based on the application timing of each illumination light, the filter changer 440 is driven so that the first and second optical filters 413 and 453 are alternately switched. Specifically, based on an application timing of the first excitation light 406, the first optical filter 413 is set. Further, based on an application timing of the second excitation light 456, the second optical filter 453 is set. When the visible light 405 is applied, the first and second optical filters 413 and 453 may not be switched.

As described above, the first and second excitation light source units 460 and 470 for applying the first and second excitation light 406 and 456 are provided, and the first optical filter 413 and the second optical filter 453 corresponding to the first excitation light source unit 460 and the second excitation light source unit 470, respectively, are provided to the camera 403. Then, based on the application timing of the illumination light, the first and second optical filters 413 and 453 are alternately switched, with the result that an image of the subject 402 that has the different types of first and second fluorescent substances 401 and 451 can be captured with high accuracy in a short exposure time.

It should be noted that the filter changer 440 may be provided with an optical filter that causes only the visible light to pass therethrough, an optical filter that causes only the first fluorescence generated from the first fluorescent substance 401 to pass therethrough, and an optical filter that causes only the second fluorescence generated from the second fluorescent substance 451 to pass therethrough. In this case, based on the application timing of the illumination light, it may be possible to capture an image of the subject 402 by alternately switching between the three filters described above. Alternatively, those three filters and the filter changer including the first and second optical filters 413 and 453 may be used. Optical filters provided to the filter changer 440 may be set as appropriate in accordance with, for example, wavelength bands of the excitation light and fluorescence of the fluorescent substances in the subject 402 or the purpose of observation of the subject 402.

### Fifth Embodiment

Description will be given on a surgical navigation system according to a fifth embodiment. In the surgical navigation system according to this embodiment, the imaging system according to each embodiment described above is used for navigation of a surgical operation. By the surgical navigation system of this embodiment, an image of an operated portion to which a fluorescent substance is given is captured.

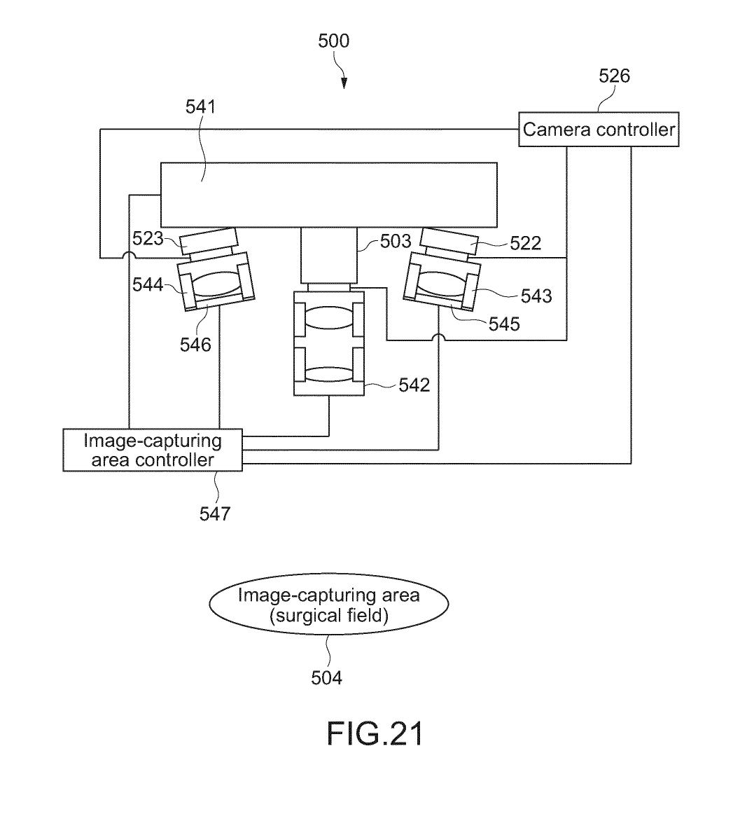

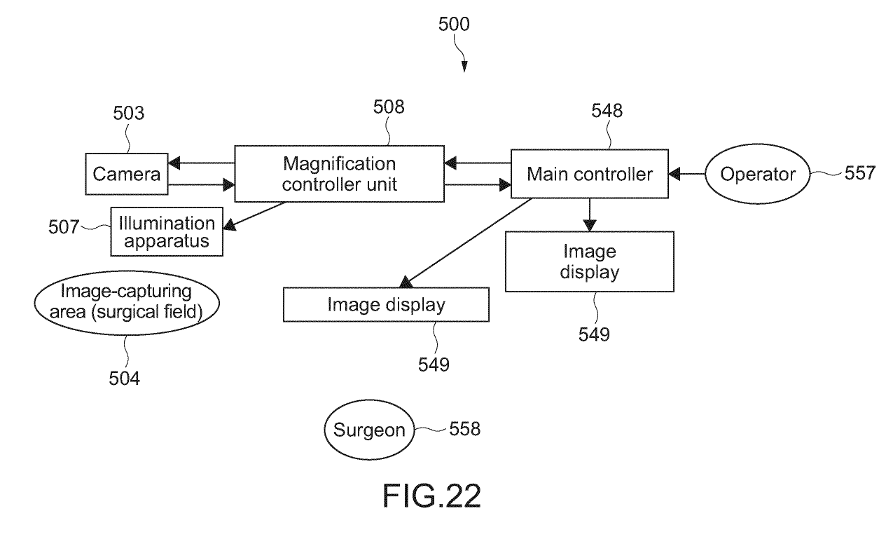

FIGS. 21 and 22 are schematic diagrams showing an of a structure of the surgical navigation system according to this embodiment. As shown in FIG. 21, a surgical navigation system 500 includes a camera 503 and a tilt stage 541 on which a visible-light illumination unit 522 and an excitation-light illumination unit 523 included in an illumination apparatus 507 are installed.

As shown in FIG. 21, the camera 503 includes a zoom lens unit 542 for image capturing, and the visible-light illumination unit 522 includes a visible-light illumination zoom lens unit 543 and the excitation-light illumination unit 523 includes an excitation-light illumination zoom lens unit 544. In this embodiment, an optical filter unit is provided in the camera 503, but the optical filter unit may be provided in front of the zoom lens unit 542 for image capturing. A visible-light filter 545 is provided in front of the visible-light illumination zoom lens unit 543, and an excitation-light filter 546 is provided in front of the excitation-light illumination zoom lens unit 544.

The tilt stage 541 and the zoom lens units 542, 543, and 544 are controlled by an image-capturing area controller 547. The image-capturing area controller 547 according to this embodiment is provided in a magnification controller unit 508 shown in FIG. 22, together with a camera controller 526. However, the image-capturing area controller 547 may be provided separately from the magnification controller unit 508.

Further, the surgical navigation system 500 of this embodiment is provided with a main controller 548 that controls the whole of the system 500 including the magnification controller unit 508. As the main controller 548, for example, a general-purpose PC is used and the PC also functions as the magnification controller unit 508.

The main controller 548 is connected with an image display 549 on which a captured image is displayed. Although the number of image displays 549 may be one, two or more image displays 549 for an operator (of controller) and a surgeon are typically provided.

An operator 557 inputs information such as instruction of a position or size of an image-capturing area (surgical field) 504 as an operated portion, or instruction of an image-capturing method. For example, the position of the image-capturing area 504 is a position of a removed portion operated by a surgeon 558. Further, examples of the image-capturing method include a method of capturing a combined image of a visible light image and a fluorescence image and a method of capturing only a visible light image in order to record the operated portion.

The instruction information is output from the main controller 548 that has received various types of instruction information to the magnification controller unit 508, and the camera 503, the visible-light illumination unit 522, the excitation-light illumination unit 523, the tilt stage 541, and the zoom lens units 542, 543, and 544 are controlled. Accordingly, the visible-light image information and the fluorescence image information of the operated portion to which the fluorescent substance has been given are output to the main controller 548, and based on those pieces of image information, a combined image of the visible light image and the fluorescence image is displayed on the image display 549.

In the surgical navigation system 500 of this embodiment, the tilt stage 541 is controlled by the image-capturing area controller 547, and accordingly a movement of the image-capturing area 504, that is, panning is enabled. In conjunction with the panning of the camera 503, illumination areas of the visible-light illumination unit 522 and the excitation-light illumination unit 523 also move, with the result that efficient illumination and image capturing can be performed.

Further, the zoom lens units 542, 543, and 544 are controlled by the image-capturing area controller 547, and accordingly the adjustment of the size of the image-capturing area 504, that is, zooming is enabled. In conjunction with the zooming of the camera 503, the size of the illumination areas of the visible-light illumination unit 522 and the excitation-light illumination unit 523 is also set as appropriate.

It should be noted that in this embodiment, a signal of the image-capturing area controller 547 is fed back to the camera controller 526. Accordingly, for example, when the size of the image-capturing area 504 is changed, in conjunction with the change, an illumination light amount of the excitation light applied by the excitation-light illumination unit 523 can be changed.

The surgeon can perform surgery efficiently and accurately while observing the combined image of the visible light image and the fluorescence image of an operated portion (image-capturing area 504) to which the fluorescent substance is given.

### Sixth Embodiment

Description will be given on an imaging apparatus according to a sixth embodiment. The imaging apparatus according to the sixth embodiment is a device in which the camera 3, the visible-light illumination unit 22, the excitation-light illumination unit 23, and the magnification controller unit 8 of the imaging system 100 shown in FIGS. 1 and 2 are integrated. For example, a control unit provided in the camera (see FIG. 3) functions as the magnification controller unit 8.

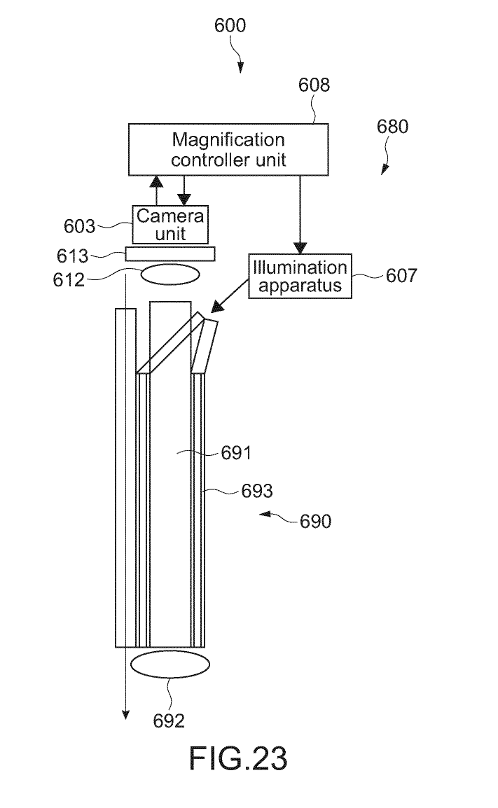

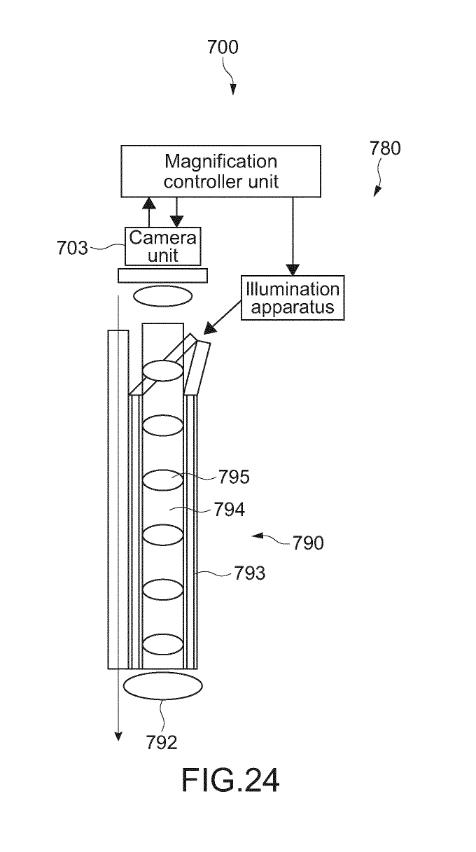

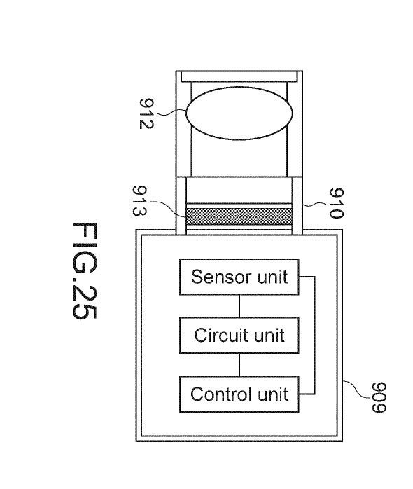

FIGS. 23 and 24 are schematic diagrams each showing an of a structure of an endoscope serving as an imaging apparatus according to this embodiment. FIG. 23 shows a flexible endoscope, and FIG. 24 shows a rigid endoscope.

The flexible endoscope 600 shown in FIG. 23 includes a main body 680 and a manipulator unit 690. The main body 680 includes a camera unit 603 serving as an imaging unit, an optical filter unit 613, a projector lens 612, and an illumination apparatus 607 including a visible-light illumination unit and an excitation-light illumination unit (that are not shown). Further, the main body 680 includes a magnification controller unit 608 that controls the camera unit 603 and the illumination apparatus 607.

The manipulator unit 690 is a portion inserted into a body, and includes an imaging fiberscope 691, for example. Though not shown, the manipulator unit 690 is provided with a device for removing or seaming an operated portion of a body, a device for extracting a tissue of a body as a specimen, or the like. In the leading end of the manipulator unit 690, an image-capturing lens 692 is arranged.

In this embodiment, in the manipulator unit 690, an illumination guide fiber 693 for guiding illumination light from the illumination apparatus 607 to an operated portion is provided around the imaging fiberscope 691. Accordingly, visible light and excitation light are alternately applied to the operated portion. The visible light reflected on the operated portion and the fluorescence generated from the fluorescent substance are incident into the camera unit 603 through the imaging fiberscope 691. Accordingly, a highly accurate visible light image and fluorescence image are captured.

The rigid endoscope 700 shown in FIG. 24 includes a main body 780 having the same structure as that of the main body 680 of the flexible endoscope 600 shown in FIG. 23, and a manipulator unit 790. The manipulator unit 790 includes an image guide shaft 794 and a plurality of relay lenses 795 inside the image guide shaft 794. Visible light and excitation light guided by an illumination guide fiber 793 are alternately applied to an operated portion via an image-capturing lens 792. The visible light and the fluorescence that have passed through the image-capturing lens 792 are guided by the plurality of relay lenses 795 to be incident into the camera unit 703.

In addition to the above, as the imaging apparatus according to this embodiment, an apparatus capable of capturing an image of a subject obtained by an optical microscope may be used. As such an apparatus, for example, a scanning apparatus having a function of an optical microscope may be used. An illumination optical system of the optical microscope is provided with a visible-light illumination unit and an excitation-light illumination unit, and based on an application timing of the illumination light, a visible light image and a fluorescence image are captured. Accordingly, an enlarged image of a subject having a fluorescent substance is captured with high accuracy in a short exposure time.

In the imaging apparatus according to this embodiment described above, a visible light image and a fluorescence image are captured by one camera unit 603 or 703, which is advantageous for the downsizing of the imaging apparatus.

### Other Embodiments

The embodiments according to the present disclosure are not limited to the embodiments described above, and other various embodiments are conceived.

For example, when a visible light image and a fluorescence image are combined, gain values of the visible light image and the fluorescence image may be adjusted independently. For example, the gain value of the visible light image is adjusted such that the image is not saturated, that is, contrast is obtained within the visible light image. The fluorescence image is adjusted based on a preset setting value at a time when an imaging system or an imaging apparatus is activated, for example.

For example, an image of a fluorescent substance is captured in a state in which a concentration and a state of conservation are managed, under the excitation-light illumination as a reference or the excitation-light illumination of the excitation-light illumination unit to be used. After that, the setting value may be determined so that the image-captured fluorescent substance has desired brightness.

The adjustment of the gain values of the visible light image and the fluorescence image may be performed by the magnification controller unit 8 or the image processing unit 27 shown in FIG. 2. Alternatively, it may be possible to structure the magnification camera unit 85 shown in FIG. 2 by giving a function of the camera controller 26 to the control unit 16 in the camera 3 and adjust gain values of both images by the magnification camera unit 85.

On the fluorescence image shown in FIG. 11, various types of image processing may be performed. For example, based on luminance values of the fluorescence image 29, image processing of indicating the distribution of the luminance values may be performed. In other words, like a thermographic image showing temperature distribution of a subject whose image is to be captured, a fluorescence image 29 may be captured. Accordingly, in the subject, the distribution of the fluorescent substance 1 based on the intensity of fluorescence can be grasped. Alternatively, threshold values may be set in advance for the luminance values of the fluorescence image 29, and only portions having larger luminance values than the threshold values may be displayed on the fluorescence image 29 as the fluorescent substance 1. Accordingly, a portion having a high intensity of fluorescence, typically, a portion to be the center of the fluorescent substance can be grasped. The image processing may be performed based not on the luminance values of the fluorescence image 29, but on the image signal read from the imaging element.

In the embodiments described above, as shown in FIG. 3, the optical filter 13 is provided in front of the lens 12 in the camera 3. As shown in FIG. 25, however, an optical filter 913 may be arranged at a camera mount 910 that is positioned after a lens 912 and in front of a main body 909.

In the embodiments described above, the visible light is divided into the three component light beams of R, G, and B. However, the visible light may be divided into three component light beams of Cyan (C), Magenta (M), and Yellow (Y), or may be divided into component light beams of three colors or more. In the case where the visible light is divided into component light beams of three colors or more, a plurality of imaging elements corresponding to the number of component light beams may be provided.

In the first embodiment, a filter changer may be used as an optical filter unit. In this case, the filter changer only has to be provided with an optical filter that causes only visible light to pass therethrough and an optical filter that causes only fluorescence generated from the fluorescent substance to pass therethrough and switch the optical filters in accordance with an application timing of the visible light and the fluorescence.

The fluorescent substance whose image is to be captured is not limited to the ICG. For example, various fluorescent substances such as DAPI (4′,6-Diamidine-2-phenylindole dihydrochloride) may be used.

In the surgical navigation system 500 described in the fifth embodiment, for , in the case where an illumination device capable of controlling an application time of an LED or the like is used as a surgical light for an operating room in which surgery is performed, the surgical light may be used as a part or all of the visible-light illumination unit.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

The application is claimed as follows:

1. An imaging apparatus, comprising:
a first illumination unit configured to apply visible light to a subject having a fluorescent substance;
a second illumination unit configured to apply excitation light to the subject so that fluorescence is generated from the fluorescent substance;
an optical filter unit configured to cause the visible light applied by the first illumination unit and the fluorescence generated from the fluorescent substance to pass therethrough, and shield the excitation light applied by the second illumination unit;
an imaging unit including:
a plurality of imaging elements capable of generating respective image signals based on incident light, and
an output unit to read the image signals from the plurality of imaging elements and output image information based on the read image signals;
an optical element configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements; and
a control means for alternately applying the visible light and the excitation light and alternately outputting image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.

2. The imaging apparatus according to claim 1, wherein
the output unit is capable of reading the image signals from the respective imaging elements in an interlace system and outputting first field image information and second field image information that constitute frame image information based on the read image signals, and
the control means outputs the image information of the visible light as the first field image information and outputs the image information of the fluorescence as the second field image information.

3. The imaging apparatus according to claim 1, wherein
the plurality of imaging elements are complementary metal oxide semiconductor (CMOS) image sensors that generate the image signals in a rolling shutter system, and
the control means stops applying the visible light and the excitation light during a time in which the image signals are being read from the CMOS image sensors by the output unit.

4. The imaging apparatus according to claim 1, wherein
the subject has a first fluorescent substance to generate first fluorescence by first excitation light being applied, and a second fluorescent substance to generate second fluorescence by second excitation light being applied, the second excitation light being different from the first excitation light,
the second illumination unit includes a first excitation-light illumination unit to apply the first excitation light, and a second excitation-light illumination unit to apply the second excitation light,
the optical filter unit includes a first filter to cause the visible light and the first fluorescence to pass therethrough and shield the first excitation light, and a second filter to cause the visible light and the second fluorescence to pass therethrough and shield the second excitation light,
the optical element divides the visible light having passed through one of the first optical filter and the second optical filter into a plurality of component light beams, causes the divided component light beams to be incident on the respective imaging elements, and causes the first fluorescence and the second fluorescence to be incident on at least one of the plurality of imaging elements,
the control means switches between the visible light, the first excitation light, and the second excitation light to be applied, alternately switches, based on a timing of the switching, between the first optical filter and the second optical filter, and alternately outputs the image information of the visible light, image information of the first fluorescence, and image information of the second fluorescence, the image information of the first fluorescence being based on at least one of the image signals read from the at least one of the imaging elements on which the first fluorescence is incident, the image information of the second fluorescence being based on at least one of the image signals read from the at least one of the imaging elements on which the second fluorescence is incident.

5. An imaging system, comprising:
an illumination apparatus including
a first illumination unit configured to apply visible light to a subject having a fluorescent substance, and
a second illumination unit configured to apply excitation light to the subject so that fluorescence is generated from the fluorescent substance;
an imaging apparatus including:
an optical filter unit configured to cause the visible light applied by the first illumination unit and the fluorescence generated from the fluorescent substance to pass therethrough, and shield the excitation light applied by the second illumination unit,
an imaging unit including:
a plurality of imaging elements capable of generating respective image signals based on incident light, and
an output unit to read the image signals from the plurality of imaging elements and output image information based on the read image signals, and
an optical element configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements; and
a control means for alternately applying the visible light and the excitation light and alternately outputting image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.

6. A surgical navigation system, comprising:
a display;
an illumination apparatus including:
a first illumination unit configured to apply visible light to an operated portion to which a fluorescent substance is given, and
a second illumination unit configured to apply excitation light to the operated portion so that fluorescence is generated from the fluorescent substance;
an imaging apparatus including:
an optical filter unit configured to cause the visible light applied by the first illumination unit and the fluorescence generated from the fluorescent substance to pass therethrough, and shield the excitation light applied by the second illumination unit,
an imaging unit including:
a plurality of imaging elements capable of generating respective image signals based on incident light, and
an output unit to read the image signals from the plurality of imaging elements and output image information based on the read image signals, and
an optical element configured to divide the visible light having passed through the optical filter unit into a plurality of component light beams, cause the divided component light beams to be incident on the respective imaging elements, and cause the fluorescence generated from the fluorescent substance to be incident on at least one of the plurality of imaging elements; and
a control means for alternately applying the visible light and the excitation light, alternately outputting image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident, and displaying a visible light image and a fluorescence image on the display based on the output image information of the visible light and the output image information of the fluorescence.

7. An imaging method, comprising:
alternately applying, to a subject having a fluorescent substance, visible light and excitation light for generating fluorescence from the fluorescent substance;
causing, an optical filter unit, the visible light applied to the subject and the fluorescence generated from the fluorescent substance to pass therethrough, and shielding the excitation light applied to the subject;
dividing the visible light having passed through the optical filter unit into a plurality of component light beams, causing the divided component light beams to be incident on a plurality of imaging elements capable of generating respective image signals based on incident light, and causing the fluorescence to be incident on at least one of the plurality of imaging elements, to thereby alternately output image information of the visible light and image information of the fluorescence, the image information of the visible light being based on the image signals read from the plurality of imaging elements on which the respective component light beams are incident, the image information of the fluorescence being based on at least one of the image signals read from the at least one of the plurality of imaging elements on which the fluorescence is incident.
